(19) United States
(12) Patent Application Publication
(10) Pub. No.: US 2022/0164861 A1
(43) Pub. Date: May 26, 2022

(54) SYSTEM AND METHOD FOR MANAGING VETERINARIAN PHARMACY TRANSACTIONS

(71) Applicant: Chewy, Inc., Dania Beach, FL (US)

(72) Inventors: Mikle Surilov, Boston, MA (US); Christopher John Prinos, Concord, MA (US); Stephen William Perry, Danvers, MA (US)

(21) Appl. No.: 17/537,360

(22) Filed: Nov. 29, 2021

Related U.S. Application Data
(63) Continuation of application No. PCT/US2020/035284, filed on May 29, 2020.
(60) Provisional application No. 62/854,596, filed on May 30, 2019.

(51) Int. Cl. G06Q 30/06; G16H 20/10; G06Q 10/08

(52) U.S. Cl. CPC G06Q 30/0641; G06Q 10/083; G06Q 10/087; G16H 20/10

(57) ABSTRACT

Some embodiments of the present invention provide improvements to traditional pharmacy systems by integrating at least some portions of a customer-facing veterinarian pharmaceutical ordering system with at least some portions of a pharmacy backend system that fulfils orders on behalf of the veterinarian thereby allowing a veterinarian to sell pharmaceutical products directly to customers without physically stocking the medication at the hospital or clinic.

### CROSS-REFERENCE TO RELATED APPLICATIONS

This application claims the benefit of U.S. Provisional Patent Application No. 62/854,596 filed May 30, 2019 entitled “System and Method for Managing Veterinarian Pharmacy Transactions”, which is incorporated by reference herein in its entirety.

### BACKGROUND OF THE INVENTION

The present invention generally relates to veterinarian pharmacy transactions and, more particularly, to a managing electronic veterinarian pharmacy transactions.

Traditionally, a pet owner brings their pet to a veterinarian at an animal hospital. After examining the animal, the veterinarian may prescribe a specific medication by writing a physical prescription for the medication. The pet owner then has to submit this prescription to a pharmacy, either hosted at the veterinarian's animal hospital or at a retail pharmacy to receive the medication. Both scenarios are problematic for the veterinarian's office for different reasons. For example, if the veterinarian hosts the pharmacy, the veterinarian must keep the medication in stock, resulting in increased overhead costs. Alternatively, if the pet owner goes to a retail pharmacy to fill the prescription, the veterinarian loses the opportunity to receive profits from sale of the medication.

Prescription renewal is another issue. To renew the prescription, the pet owner has to revisit the veterinarian. This traditional method of prescribing medication has led to non-compliance for various reasons. Amongst the various reasons of non-compliance is having to revisit the veterinarian to renew the prescription and then re-processing the prescription at a pharmacy. This process can be time consuming for the pet owner. A new system and method for managing veterinarian pharmacy transactions is described herein.

### BRIEF SUMMARY OF THE INVENTION

In one embodiment, there is a computer-implemented method, including populating a database, at a backend computing system, first product data identifying one or more products for display on a veterinarian customer-facing user interface, the first product data including, for each product identified in the first product data, product identifier, product name, product wholesale price. The method further includes transmitting, from the backend computing system, the first product data to a veterinarian computing system, and for each respective product identified in the first product data creating, at the veterinarian computing system, a respective product record at a veterinarian database at the veterinarian computing system. Each respective product record includes the product identifier, the product name, a product retail price indicative of a price of the respective product on the veterinarian customer-facing user interface, and a product offering flag indicative of whether the veterinarian intends to offer the respective product on the veterinarian customer-facing user interface, displaying, at a veterinarian administrative user interface of the veterinarian computing system, for each respective product record, a respective administrative product display item. The respective administrative product display item includes the product name, a product price field populated with the product retail price of the corresponding product record, and a product offering field associated with the product offering flag and indicative of whether the veterinarian intends to offer the product on the veterinarian customer-facing user interface. The method further includes receiving, at the veterinarian administrative user interface, a user input at the product offering field of the respective administrative product display item, determining whether the user input at the product offering field for the respective administrative product display item indicates user intent to offer the respective product on the veterinarian customer-facing user interface and in response to determining the user intends to forego offering the product on the veterinarian customer-facing user interface, updating the product offering flag in the corresponding product record to indicate that the veterinarian intends to forego offering the product on the veterinarian customer-facing user interface.

In some embodiments, the method further includes receiving a request for displaying the veterinarian customer-facing user interface. For each respective product record, the method may include determining whether the respective product record includes the product offering field indicating user intent to offer the product, in response to a determination that the respective product record includes the product offering field indicating user intent to offer the product, updating a veterinarian customer-facing user interface file representative of the veterinarian customer-facing user interface to include a customer product display item. The customer product display item includes the product name, and a product price field populated with the product retail price of the corresponding product record. Further, the method may include, in response to a determination that the respective product record does not include the product offering field indicating user intent to offer the product, forego updating a veterinarian customer-facing user interface file representative of the veterinarian customer-facing user interface to include the customer product display item.

In some embodiments, the method further includes receiving, at the backend computing system, second product data identifying one or more products for display on the veterinarian customer-facing user interface, transmitting, at the backend computing system, the second product data to the veterinarian computing system, and for each respective product identified in the second product data, creating, at a veterinarian computing system, a respective product record at the veterinarian database at the veterinarian computing system.

In some embodiments, the method further includes receiving, at the veterinarian administrative user interface, a user input at the product price field of the respective administrative product display item, the user input including a price indicative of the price the veterinarian intends to sell the product on the veterinarian customer-facing user interface, and updating the product price in the corresponding product record to the price indicated in the product price field.

In some embodiments, the product retail price is initially set to the product wholesale price of the respective product.

In some embodiments, the method further includes a system including one or more memory units each operable to store at least one program and at least one processor communicatively coupled to the one or more memory units, in which the at least one program, when executed by the at least one processor, causes the at least one processor to perform the steps of the method

Another embodiment of the present invention provides for a computer-implemented method, including displaying, at an order submission computing system, a pharmaceutical ordering user interface including pharmaceutical product items identifying one or more pharmaceutical products for ordering by a user, receiving, at the order submission computing system, pharmaceutical ordering information representative of one or more products ordered by the user, the pharmaceutical ordering information including at least: product information, product quantity and user identification, transmitting, from the order submission computing system to a backend computing system, the pharmaceutical ordering information, and determining whether the user identification of the pharmaceutical ordering information indicates that the user is authorized to approve a prescription. The method further includes in response to determining that the user identification of the pharmaceutical ordering information indicates that the user is not authorized to approve a prescription, transmitting, from the backend computing system to a prescription approval computing system, a prescription order approval user interface requesting approval of the pharmaceutical product from an authorized veterinarian. The method further includes in response to determining that the user identification of the pharmaceutical ordering information indicates that the user is authorized to approve a prescription, refrain from transmitting, by the backend computing system to the prescription approval computing system, the prescription order approval user interface. The method further includes receiving, at the prescription approval computing system, prescription order approval information indicative of approval of the pharmaceutical order submitted by the ordering authorized user, transmitting, from the prescription approval computing system to the backend computing system, the prescription order approval information, receiving, at an order management subsystem of the backend computing system, the pharmaceutical ordering information and transmitting, from the order management subsystem to a retail pharmacy computing system, the pharmaceutical ordering information, and receiving, at the retail pharmacy subsystem, the prescription order approval information from the prescription approval computing system. The method further includes in response to a transfer request received from an administrator using the wholesale pharmacy subsystem, transferring the pharmaceutical product from a wholesale site to a retail site; and fulfilling and shipping the pharmaceutical product to an address identified in the pharmaceutical ordering information.

In some embodiments, the method further includes receiving, at the wholesale pharmacy subsystem, the pharmaceutical ordering information including the product information and veterinarian identification information, wherein the product information includes: product name information and product quantity information and creating a wholesale database, at the wholesale pharmacy subsystem, a wholesale pharmaceutical order record including: the veterinarian identification information, the product quantity and the product name information.

In some embodiments, the method further includes populating a wholesale database with a respective product wholesale inventory record for one or more pharmaceutical products, wherein for each pharmaceutical product, the respective product wholesale inventory record including the product name and a product wholesale inventory count, and populating a retail database with a respective product retail inventory record for one or more pharmaceutical products, wherein for each pharmaceutical product, the respective product retail inventory record including the product name and a product retail inventory count. The method further includes after transferring an amount of the pharmaceutical product from a wholesale site to a retail site in response to a transfer request using the wholesale pharmacy subsystem, updating the respective product wholesale inventory record by decrementing the product wholesale inventory count by the amount of the transferred pharmaceutical product and updating the respective product retail inventory record by incrementing the product retail inventory count by the amount of the transferred pharmaceutical product.

In some embodiments, the method further includes determining whether the user identification of the pharmaceutical ordering information indicates that the user is a ordering authorized user includes comparing the user identification in the pharmaceutical ordering information to one or more veterinarian records in a veterinarian database. The method further includes in response to detecting a match of the user identification to one of the veterinarian records, determining that the user identification of the pharmaceutical ordering information indicates that the user is authorized to approve a prescription and in response to failing to detecting a match of the user identification to one of the veterinarian records, determining that the user identification of the pharmaceutical ordering information indicates that the user is not authorized to approve a prescription.

In some embodiments, the method further includes, before transferring the pharmaceutical product from a wholesale site to a retail site in response to a transfer request from an administrator using the wholesale pharmacy subsystem displaying a warehouse inventory transfer user interface including one or more pharmaceutical product order items. Each warehouse pharmaceutical product order item includes a pharmaceutical product name, a veterinarian identification indicating the veterinarian associated with a respective pharmaceutical product order, a pharmaceutical quantity, and a transfer approval affordance that, when selected by the administrator, generates the transfer request from the administrator.

In some embodiments, the prescription order approval information comprises authorization information unique to the authorizing veterinarian.

In some embodiments, pharmaceutical ordering information includes auto-shipment information.

In some embodiments, auto shipment information comprises a number of future auto shipments and at least one of a frequency of auto shipments and date information.

In some embodiments, the method further includes creating an order associated with a single auto shipment event at a predetermined time relative to a schedule associated with the auto shipment event, and delaying creating a next order associated with a future single auto shipment event until a predetermined time relative to a schedule that the future auto shipment is due to be made.

In some embodiments, the method further includes creating an electronic consignment record indicative of a wholesale quantity of the one or more pharmaceutical products and a retail quantity of the one or more pharmaceutical products, the retail quantity reflecting a quantity associated with an order.

In some embodiments, the method further includes transmitting, from the retail pharmacy subsystem to a wholesale pharmacy subsystem, the pharmaceutical ordering information, wherein the retail subsystem is separate and distinct from the wholesale pharmacy subsystem and in response to a transfer request received at the wholesale pharmacy subsystem, transferring the pharmaceutical product record from a wholesale site to a retail site.

In some embodiment, the method further includes a system including one or more memory units each operable to store at least one program and at least one processor communicatively coupled to the one or more memory units, in which the at least one program, when executed by the at least one processor, causes the at least one processor to perform the steps of the method.

### BRIEF DESCRIPTION OF THE SEVERAL VIEWS OF THE DRAWINGS

The foregoing summary, as well as the following detailed description of embodiments of the system and method for managing veterinarian pharmacy transactions, will be better understood when read in conjunction with the appended drawings of an exemplary embodiment. It should be understood, however, that the invention is not limited to the precise arrangements and instrumentalities shown.

In the drawings:

FIG. 1 is an block diagram illustrating an implementation of a veterinarian pharmacy transaction system, in accordance with an exemplary embodiment of the present invention.

FIGS. 2A-2E are exemplary user interfaces of a veterinarian pharmacy transaction system, in accordance with an exemplary embodiment of the present invention.

FIGS. 3A-3B are conceptual block diagrams illustrating a sequence for an implementing of a veterinarian pharmacy transaction system, in accordance with an exemplary embodiment of the present invention.

FIGS. 4A-4C illustrate exemplary user interfaces for submitting an order for a prescription product, in accordance with an exemplary embodiment of the present invention.

FIGS. 4D-4E illustrate exemplary user interfaces for approving a prescription for a prescription product, in accordance with an exemplary embodiment of the present invention

FIGS. 5A-5B illustrate exemplary user interfaces for transferring a desired amount of a prescription product, in accordance with an exemplary embodiment of the present invention.

FIGS. 6A-6C illustrate exemplary flow charts representing a method for submitting an order for a prescription product, in accordance with an exemplary embodiment of the present invention.

### DETAILED DESCRIPTION

Numerous details are described herein in order to provide a thorough understanding of the example embodiments illustrated in the accompanying drawings. However, some embodiments may be practiced without many of the specific details, and the scope of the claims is only limited by those features and aspects specifically recited in the claims. Furthermore, well-known methods, components, and circuits have not been described in exhaustive detail so as not to unnecessarily obscure pertinent aspects of the embodiments described herein.

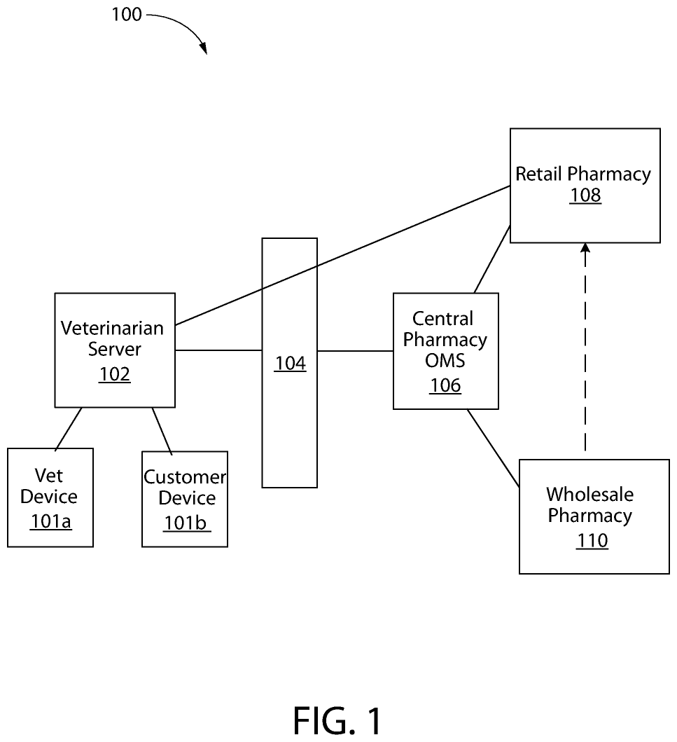

Some embodiments of the present invention provide improvements to traditional pharmacy systems by integrating at least some portions of a customer-facing veterinarian pharmaceutical ordering system with at least some portions of a pharmacy backend system that fulfils orders on behalf of the veterinarian thereby allowing a veterinarian to sell pharmaceutical products directly to customers without physically stocking the medication at the hospital or clinic. Referring to the drawings in detail, wherein like reference numerals indicate like elements throughout, there is shown in FIGS. 1-3B a veterinarian pharmacy transaction system, generally designated, in accordance with an exemplary embodiment of the present invention.

Referring to FIG. 1, there is shown a block diagram illustrating an implementation of a veterinarian pharmacy transaction system 100, generally designated. While some example features are illustrated, various other features have not been illustrated for the sake of brevity and so as not to obscure pertinent aspects of the example embodiments disclosed herein. To that end, as a non-limiting example, the veterinarian pharmacy transaction system 100, as illustrated in FIG. 1, comprises of a veterinarian server 102, application platform interface (API) 104, central pharmacy order management system (OMS) 106, retail pharmacy server 108, and wholesale pharmacy server 110.

The veterinarian server 102 may be one or more computing servers that execute server-side portions of the application software of the invention. Veterinarian server may provide a user device 101 (e.g., veterinarian device 101a, and/or customer device 101b) with secure access to pharmaceutical product data located in a database on veterinarian server 102. User device 101 may be operated on any suitable computer device, such as a computer, a laptop computer, a tablet device, a netbook, an internet kiosk, a personal digital assistant, a mobile phone, a smart phone, a gaming device, a computer server, or any other computing device. The veterinarian server 102 may be configured to generate a user interface (or website) accessible via network (such as the internet) and viewable by a user (e.g., customer, veterinarian, clinic or hospital staff and/or other approved authorized users) on a user device 101.

The veterinarian server 102 may be configured to communicate with other pharmacy system(s) (e.g., central pharmacy OMS 106 via API 104) to receive a list of products from the other pharmacy system. In some embodiments, the veterinarian server 102 may be integrated with central pharmacy OMS 106. The veterinarian server 102 may be configured to allow a veterinarian to select products to display to a customer as well as change other product characteristics regarding each product, such as price and quantity. For example, server 102 may be configured to customize a product display for a particular customer or a particular class of customers (e.g., customers with a particular pet, a particular species of pet or a particular condition). Thereby, customers may view products that a veterinarian may consider to be of particular interest to the customer based on, for example, the veterinarian's previous treatment or examination of pet. Veterinarians, clinics or hospitals may have full control over the products that remain published in the product catalogue on the display user interface accessed by veterinarian device 101a and/or customer device 101b. Veterinarians, clinics and hospitals may set their own prices for products in the product catalogues on the veterinarian server 102. The prices of products in the product catalogue of the veterinarian server 102 may be set to prices that are lower than or higher than the default prices in the product catalogue of the central pharmacy OMS 106. This may give veterinarians, hospitals or clinics the opportunity to increase or decrease a profit from the orders placed. The veterinarian server 102 may be configured to store the products selected by the veterinarian in a database and/or store an indication in the databases that the veterinarian has selected a particular product for a particular customer or class of customers.

The veterinarian server 102 may be configured to provide a display user interface for display of the one or more products (e.g., pharmaceutical products) on the veterinarian device 101a and/or customer device 101b. In some embodiments, the products displayed are only those products designated by the veterinarian to be displayed that are associated with a particular customer or class of customers. The veterinarian server 102 may be configured to receive an order for one or more products from the veterinarian device 101a and/or customer device 101b.

Once an order is placed on the veterinarian server 102, the veterinarian server 102 may be configured to send the order and/or prescription information to the API 104. API 104 may include an interface communication device configured to transmit data communications between the veterinarian server 102 and at least one of: the central pharmacy OMS 106, the wholesale pharmacy server 110 and the retail pharmacy server 108. In some embodiments, direct communications between veterinarian server 102 and retail pharmacy 108 is prevented or blocked. In some embodiments, veterinarian server 102 is configured to only communicate to Retail Pharmacy 108 through Central Pharmacy OMS 106. In some embodiments, the API 104 is a business-to-business (B2B) API that allows a central pharmacy to partner with veterinarians, clinics or hospitals to allow the veterinarians, clinics or hospitals to directly and easily place orders through a website site configured in a manner that is specific to the veterinarians, clinics and/or hospitals. The specific manner may include personalized branding (also referred to herein as a “white label site”). In some embodiments, API 104 includes one or more processing units (sometimes herein called CPUs, processors, or hardware processors, and sometimes implemented using microprocessors, microcontrollers, or the like) configured to execute instructions in one or more programs (e.g., in veterinarian server 102). The API 104 may be configured to transmit orders from the veterinarian server 102. API may be configured (e.g., in concert with veterinarian server 102 to direct orders to one or more of central pharmacy OMS 106, wholesale pharmacy server 110 or retail pharmacy server 108. The order direction (e.g., whether to central pharmacy OMS 106, wholesale pharmacy 110 or retail pharmacy 102) may be specifically set based on the particular veterinarian, veterinarian practice, class of customer, product being ordered, class of products being ordered (e.g., controlled or uncontrolled products) or some other characteristic selectable by a system administrator. In some embodiments where the wholesale pharmacy server 110 receives orders, the wholesale pharmacy server 110 may consign orders to retail pharmacy server 108 for fulfilment and shipping.

The central pharmacy OMS 106 may be include one or more computing servers that are configured to receive the orders from the API 104 and process the orders for fulfillment by the retail pharmacy server 108 and/or wholesale pharmacy server 110. In some embodiments the central pharmacy OMS 106 may be configured to generate a separate customer-facing user interface that allows customers to order products separately from the ordering system of the veterinarian server 102 as described herein.

The retail pharmacy server 108 may be one or more computing servers that may receive the order from the central OMS 106. The retail pharmacy server 108 may receive the prescription electronically from the API 104. The retail pharmacy server 108 may match the prescription with the order received from the wholesale pharmacy server 110 to confirm that each order for a pharmaceutical product requiring a prescription has an approved prescription.

The retail pharmacy server 108 may process the physical shipment to the customer. After shipment, an order shipment notification may be transmitted to the customer who placed the order.

The retail pharmacy server 108 may receive products from wholesale pharmacy server 110. The wholesale pharmacy server 110 may include one or more computing servers that may process the order received from the central pharmacy OMS 106 and, via a user interface, receive requests for transfer, and track physical transfers of, products required for the order from one or more physical wholesale pharmacy locations (e.g., wholesale pharmacy warehouse) to one or more physical retail pharmacy location (e.g., retail pharmacy warehouse).

The retail pharmacy inventory may physically reside in a different fulfillment center than the wholesale pharmacy inventory. The wholesale pharmacy server 110 may transfer product inventory to the retail pharmacy server 108 on behalf of an order placed which may be placed by a veterinarian, for fulfillment to the pet owner. Inventory consigned from wholesale pharmacy server 110 to retail pharmacy server 108 may be tracked as owned by the veterinarian whose consignment order purchased the inventory. Inventory owned by the veterinarian (e.g., indicated in a database as being owned by the veterinarian in the retail pharmacy server 108 may be unavailable as general inventory on the website of the central pharmacy OMS 106.

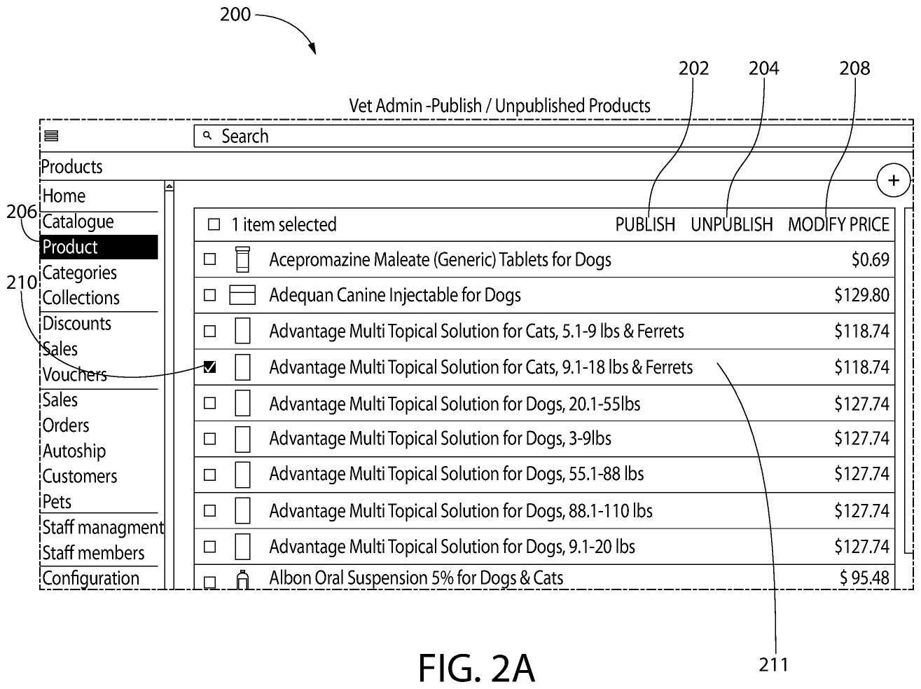

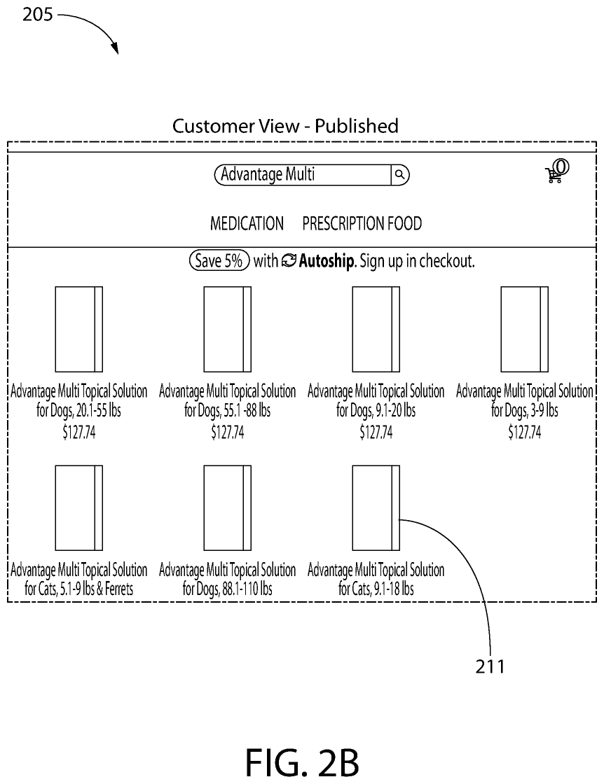

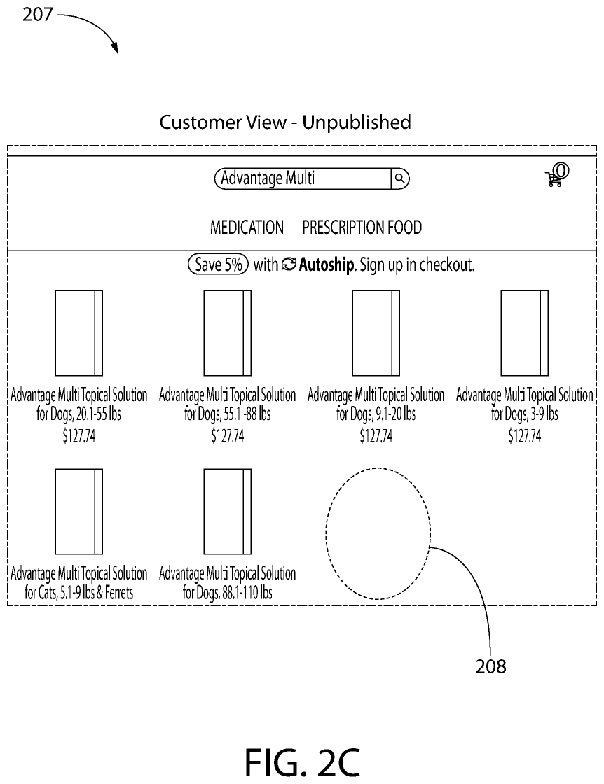

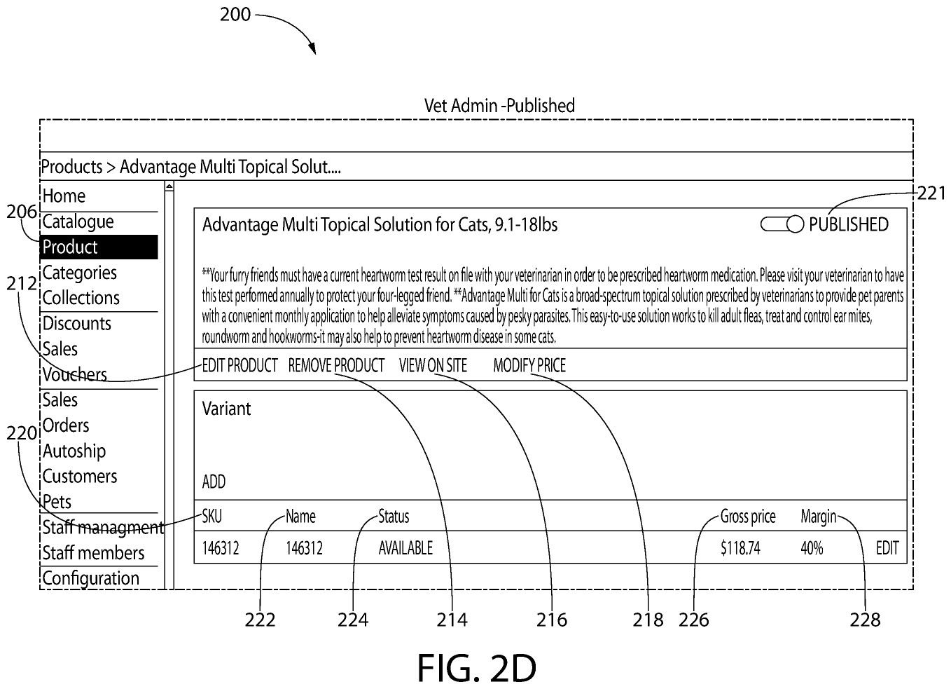

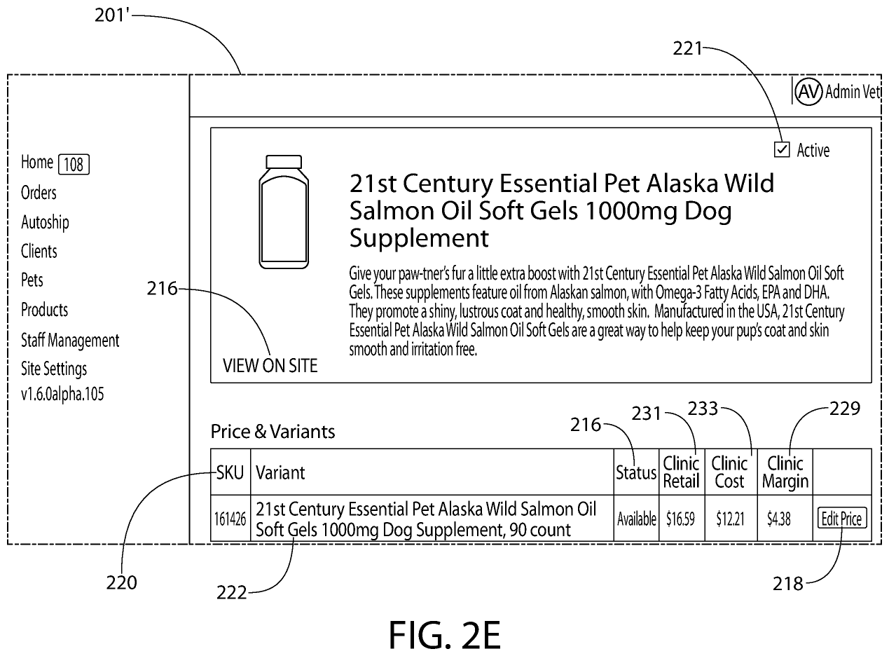

Referring to FIGS. 2A-2E, there are shown exemplary user interfaces for allowing a veterinarian to modify products and product characteristics listed on the veterinarian customer-facing website displayed to a customer. As shown in FIG. 2A, a veterinarian view of product catalogue 206 may allow veterinarians to publish or unpublish and/or modify prices 208 of any of the one or more products of product catalogue 206 supplied by the central pharmacy OMS 106. The product catalogue 206 may be based on product data transmitted from central pharmacy OMS 106. The product data may include characteristics about one or more products, such as product identifier, product name, and product wholesale price.

In some embodiments, a veterinarian may publish a product to the veterinarian's customer-facing website by selecting a product-selection affordance 210 (e.g., a checkbox) associated with a product 211, (e.g., advantage multi topical solution for cats, 9.1-18 lbs.) and then selecting the publish affordance 202. By selecting the publish affordance 202 after selecting a product 211, the product 211 may be published to the veterinarian customer-facing website displayed to a customer. FIG. 2B shows an exemplary customer user interface 205 of the veterinarian customer-facing website displayable to the customer after the product 211 has been selected for publication by the veterinarian. As shown in FIG. 2B, the product 211 (e.g., advantage multi topical solution for cats, 9.1-18 lbs.) is viewable.

In some embodiments, a veterinarian may unpublish a product from the veterinarian's customer-facing website by selecting a product-selection affordance 210 (e.g., a checkbox) associated with a product 211, (e.g., advantage multi topical solution for cats, 9.1-18 lbs.) and then selecting the unpublish affordance 204. By selecting the unpublish affordance 204 after selecting a product 211, the product 211 may be unpublished to the veterinarian customer-facing website displayed to a customer. FIG. 2C shows an exemplary user interface 207 of the veterinarian customer-facing website displayable to the customer after the product 211 has been selected for non-publication by the veterinarian. As shown in FIG. 2C, and as represented by a circle around prices 208 at a location where product 211 would be displayed, if selected, the product 211 (e.g., advantage multi topical solution for cats, 9.1-18 lbs.) selected in FIG. 2A is no longer viewable.

Referring to FIG. 2D, there is shown a veterinarian administrative view of the published product user interface 201. The veterinarian view is displayable after selection of a product 211 in FIG. 2A. Veterinarian administrative view of the published product user interface 201 may allow a user accessing the veterinarian server 102 and selecting a product (e.g., product 211) in the product catalogue 206 of FIG. 2A to access further information on the product. As shown in FIG. 2D, after clicking on product 211, the veterinarian may be able to view the published view of product 211, including product information item 227. Product information item 227 may allow a veterinarian to edit product information, remove product from being available in the product catalogue 206 of the veterinarian customer-facing website, view product on central pharmacy OMS 106, modify price of the product and/or also have the option to publish or unpublish the product. By modifying the price of the product, the veterinarian is able to profitably manage the veterinary pharmacy transactions by receiving the difference between the price selected by the veterinarian and the wholesale price set as the default for the product. Also shown in veterinarian administrative view of the published user interface 201 is a brief description of the product including SKU number 220, product name 222, status of inventory 224 (e.g., available or unavailable), gross price 226 and margin percentage 228. In some embodiments, published product user interface 201 may include options to allow a user to edit the product, remove the product, view product 211 on the website, and modify or edit the price of product 211. For example, published product user interface 201 may include edit product affordance 212, remove product affordance 214, review on site affordance 215, and modify (or edit) price affordance 218.

Referring to FIG. 2E, there is shown a veterinarian administrative view of the published product user interface 203. In some embodiments, veterinarian administrative view of the published product user interface 201′ is similar to veterinarian administrative view of the published product user interface 201, except published user interface 201′ may display an image of product 211. Veterinarian administrative view of the published product user interface 201′ may allow a user accessing the veterinarian server 102 and selecting a product (e.g., product 211) in the product catalogue 206 of FIG. 2A to access further information on the product. As shown in FIG. 2E, after clicking on product 211, the veterinarian may be able to view the published view of product 211, including product information item 227. Product information item 227 may allow a veterinarian to edit product information, remove product from the product catalogue 206 of the veterinarian customer-facing website, view product on central pharmacy OMS 106, modify price of the product and/or also have the option to mark product 211 as “Active”. For example, published product user interface 201′ may include a product offering field (e.g., publish affordance 221′). In some embodiments, by modifying the price of the product, the veterinarian is able to profitably manage the veterinary pharmacy transactions by receiving the difference between the price selected by the veterinarian and the wholesale price set as the default for the product. Also shown in veterinarian administrative view of the published user interface 201′ is a brief description of the product including SKU number 220, product name 222, and status of inventory 224 (e.g., available or unavailable). In some embodiments, published user interface 201′ may include clinic retail amount 231, and clinic cost amount 233, and margin price 229.

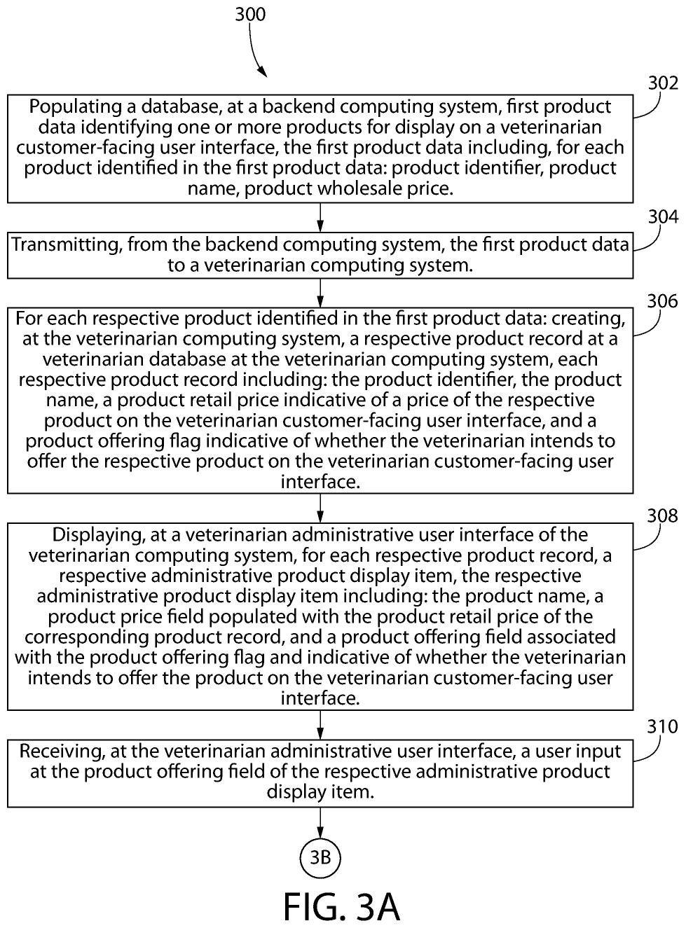

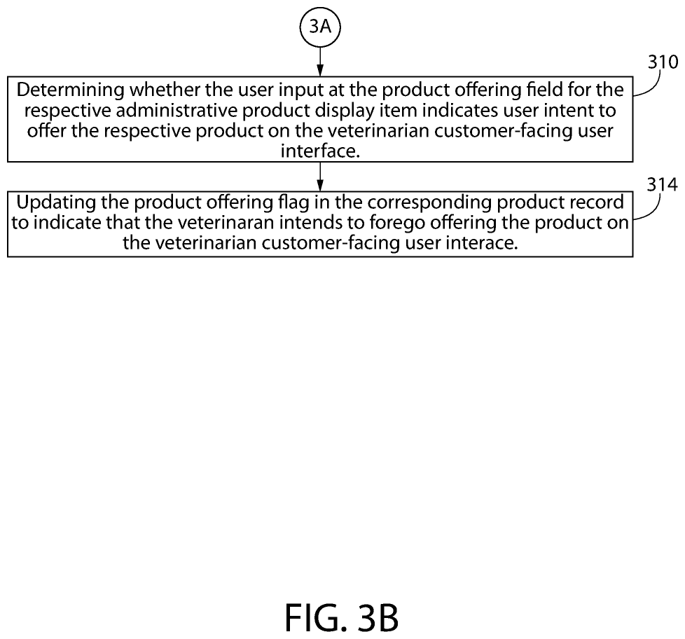

FIG. 3A, depicts a flowchart of a method 300 for implementing a veterinarian pharmacy transaction system. Method 300 is typically performed by one or more components described herein, but alternatively may be performed by other computer systems.

In one embodiment, method 300 may comprise populating (302) a database, at a backend computing system (e.g., central pharmacy OMS 106), first product data identifying one or more products for display on a veterinarian customer-facing user interface (e.g., customer device 101b), the first product data including, for each product identified in the first product data: product identifier, product name and product wholesale price.

In one embodiment, method 300 may comprise transmitting (304), from the backend computing system (e.g., central pharmacy OMS 106), the first product data to a veterinarian computing system (e.g., veterinarian server 102).

For each respective product identified in the first product data: method 300 may comprise creating (306), at the veterinarian computing system (e.g., veterinarian server 102), a respective product record (e.g., product record of product 211) at a veterinarian database at the veterinarian computing system, each respective product record including: the product identifier, the product name, a product retail price (e.g., gross price 226) indicative of a price of the respective product on the veterinarian customer-facing user interface, and a product offering flag indicative of whether the veterinarian intends to offer the respective product on the veterinarian customer-facing user interface.

In one embodiment, method 300 may comprise displaying (308), at a veterinarian administrative user interface (and displayable on a veterinarian device 101a) of the veterinarian computing system (veterinarian server 102), for each respective product record, a respective administrative product display item, the respective administrative product display item including: the product name, a product price field populated with the product retail price of the corresponding product record, and a product offering field associated with the product offering flag and indicative of whether the veterinarian intends to offer the product on the veterinarian customer-facing user interface (for example on customer device 101b). Exemplary characteristics of product 211 are shown in FIG. 2D.

In one embodiment, method 300 may comprise receiving (310), at the veterinarian administrative user interface (e.g., an administrative user interface on veterinarian device 101a), a user input at the product offering field (e.g., publish affordance 221) of the respective administrative product display item.

In one embodiment, method 300 may comprise determining (312) whether the user input at the product offering field (e.g., publish affordance 221) for the respective administrative product display item indicates user intent to offer the respective product on the veterinarian customer-facing user interface.

In one embodiment, method 300 may comprise in response to determining the user intends to forego offering the product on the veterinarian customer-facing user interface (e.g., a user interface displayable on customer device 101b), updating (314) the product offering flag in the corresponding product record to indicate that the veterinarian intends to forego offering the product on the veterinarian customer-facing user interface.

In one embodiment, the method 300 may comprise receiving a request for displaying the veterinarian customer-facing user interface (e.g., a user interface displayable on customer device 101b); for each respective product record: determining whether the respective product record includes the product offering field indicating user intent to offer the product, in response to a determination that the respective product record includes the product offering field indicating user intent to offer the product, updating a veterinarian customer-facing user interface file representative of the veterinarian customer-facing user interface to include a customer product display item including: the product name, and a product price field populated with the product retail price of the corresponding product record; and in response to a determination that the respective product record does not include the product offering field indicating user intent to offer the product, forego updating a veterinarian customer-facing user interface file representative of the veterinarian customer-facing user interface to include the customer product display item.

In one embodiment, the method 300 may comprise receiving, at the backend computing system, second product data identifying one or more products for display on the veterinarian customer-facing user interface (e.g., a user interface displayable on customer device 101b); transmitting, at the backend computing system, the second product data to the veterinarian computing system (e.g., veterinarian server 102); for each respective product identified in the second product data: creating, at a veterinarian computing system (e.g., veterinarian server 102), a respective product record at the veterinarian database at the veterinarian computing system. In some embodiments, by mimicking the product catalogs and the database attributes (e.g., product name, price, etc.) of the databases at the veterinarian server 102 and the central pharmacy OMS 106 to be substantially similar except for, e.g., a display selection field that tracks whether an authorized user of the veterinarian server 102 intends to display a particular product on the veterinarian customer-facing user interface, updates to the database at the veterinarian server 102 are faster as compared to database configurations where the two databases are not substantially similar. For example, database configurations where the two databases are not substantially similar generally require database schemas with unpopulated fields to map different attributes between the two databases and processes to match records and populate attributes between the two databases.

In one embodiment, the method 300 may comprise receiving, at the veterinarian administrative user interface (e.g., administrative user interface on veterinarian device 101a) a user input at the product price field of the respective administrative product display item, the user input including a price indicative of the price the veterinarian intends to sell the product on the veterinarian customer-facing user interface (displayable on customer device 101b); and updating the product price in the corresponding product record to the price indicated in the product price field.

In one embodiment, the product retail price (e.g., as displayed at gross price 226) is initially set to the product wholesale price (set by central pharmacy OMS 106) of the respective product (e.g., product 211).

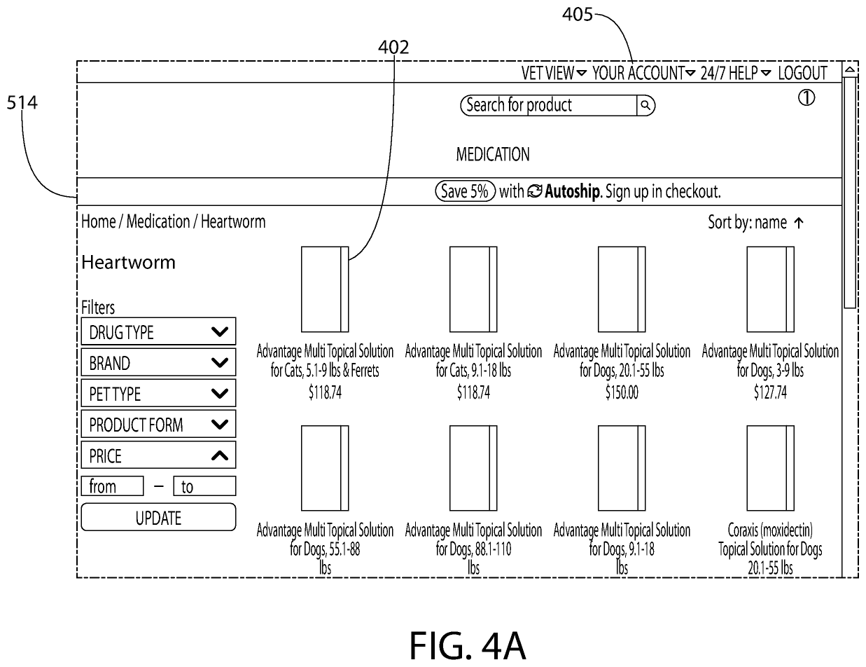

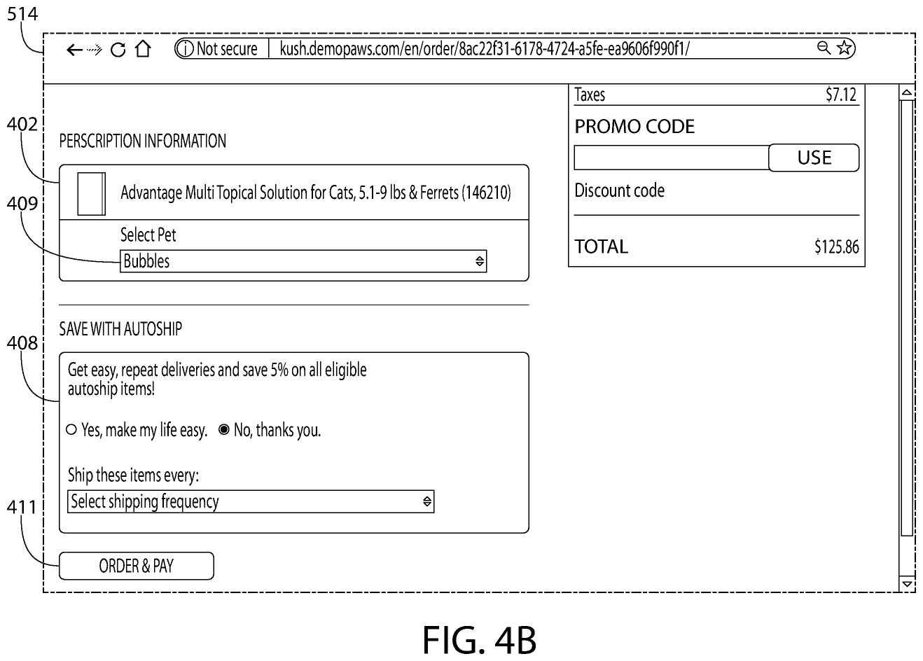

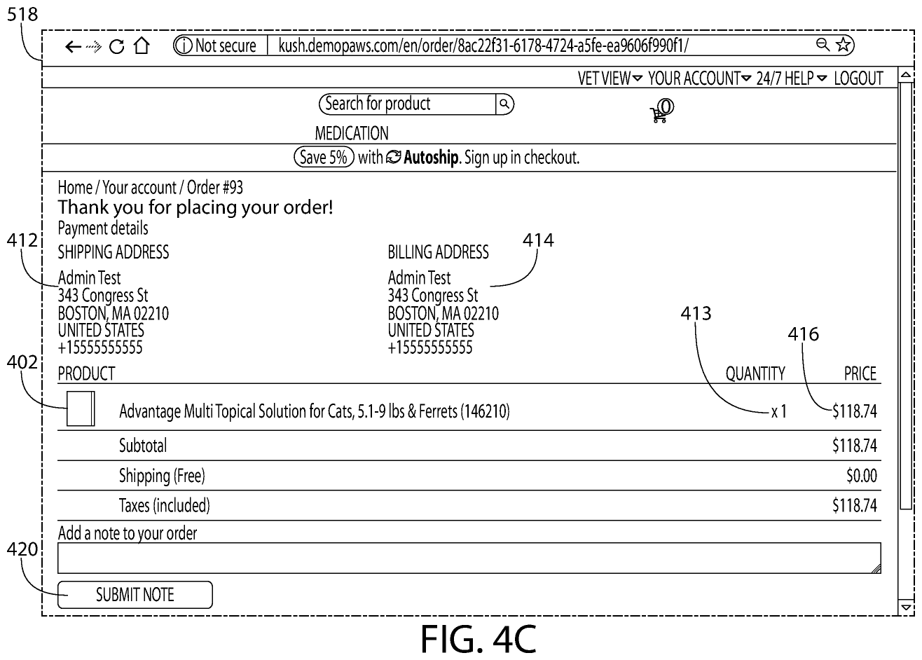

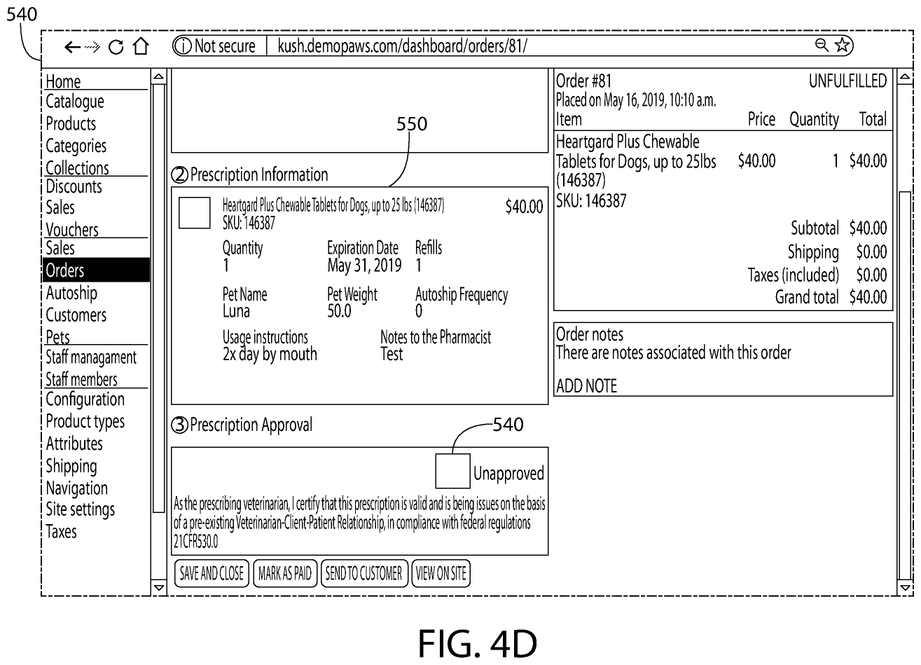

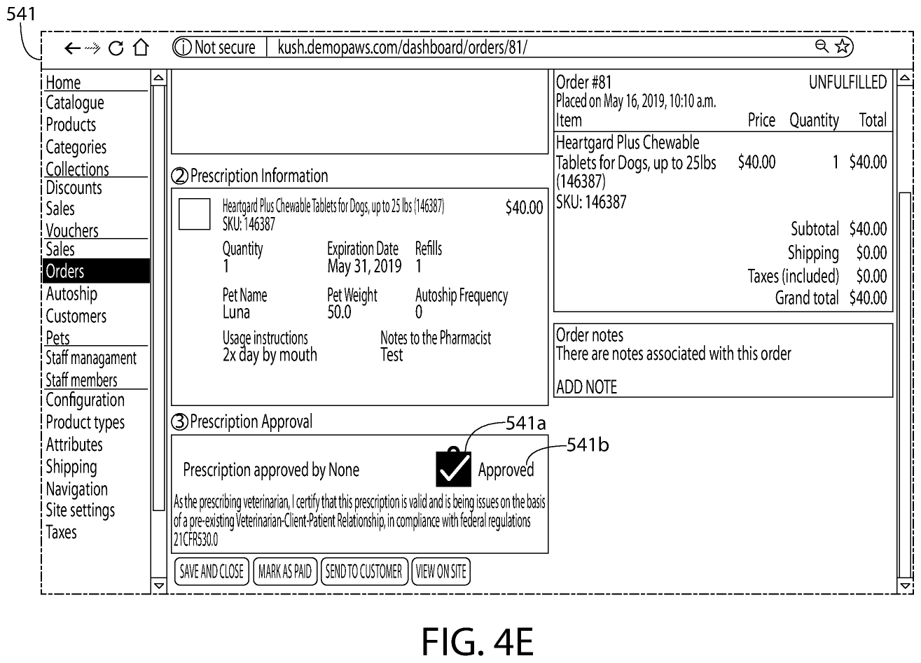

FIGS. 4A-4C illustrate exemplary user interfaces of the present invention to that are configured to submit an order for a prescription product. FIGS. 4A-4C illustrate exemplary product list user interface 514, which may be displayed on a display of an electronic device. Product list user interface 514 may be displayed on a desktop computer, laptop, tablet, or a mobile device. Product list user interface 514 may be displayed via a web browser. For example, a user may navigate to a specific website and be displayed product list user interface 514. Product list user interface 514 may include a platform for a user to create an account and purchase prescription products. For example, a user may create a username and password to access product list user interface 514, and may purchase a prescription product. In some embodiments, the prescription product is a prescription for a pet or animal, such as medicine for a dog. In some embodiments, the end user is a customer owning a pet. In some embodiments, the end user is a veterinarian. In some embodiment, the customer may be associated with a veterinarian.

Referring to FIG. 4A, product list user interface 514 may include at least one prescription product 402. Product list user interface 514 may include an array of prescription products 402 for display, or may include only one prescription product 402 for display. Prescription product 402 may be prescription products for use by an animal. For example, as shown in FIG. 4A, prescription product 402 may be medicine for a dog. Product list user interface 514 may include account information option 405. End user may create an account with a username and password to access user interface 514. Account information option 405 may be selected by the end user to view information about end user's account. In selecting account information option 405, a user may update their email address, contact information, shipping information, billing information, password, or pet information. For example, the end user may associate their pets with their account. In an embodiment, the end user's account include information about all of the end user's pets, including their name and type of pet. In some embodiments, the user may update account information to specify that the user is ordering authorized user. In some embodiments, the user may specify that the user is an ordering authorized user at account initialization rather than through the account update process.

As illustrated in FIG. 4B, selection confirmation user interface 516 may be used to confirm that the user has selected, and intends to purchase, a desired prescription product 402. Selection confirmation user interface 516 may include repeat delivery option 408. Repeat delivery option 408 may allow a user to select the frequency at which prescription product 402 will be automatically delivered. For example, a user may select every two weeks as the selection for repeat delivery option 408, therefore prescription product would be automatically delivered to the user every two weeks. Once the user has selected the desired input within repeat delivery option 408, the user may select the “Order & Pay” button 411 to complete purchase of prescription product 402 and proceed to order confirmation user interface 518. In an embodiment, where the user is not a veterinarian, prior to completing the purchase of prescription product 402, a notification may be sent to a veterinarian associated with the pet selected in pet selection field 409. The notification may require input from the veterinarian to approve the purchase of prescription product 402. If the veterinarian approves the purchase of prescription product 402 by the user for the pet selected in pet selection field 409, then the user may complete the purchase of prescription product 402 within selection confirmation user interface 516. However, if the veterinarian does not approve the purchase of prescription product 402 by the user for the pet selected in pet selection field 409, then the user may be notified that the purchase of prescription product 402 cannot be completed. In some embodiments, veterinarian approval of a customer order is completed seamlessly from the customer's perspective. A customer may complete an order placement and only upon completion of the order will a veterinarian approval be completed. Once completed, the order may be shipped to the customer automatically with no further customer input required. In some embodiments, a veterinarian approval will trigger an order completion routine that includes completion of a commercial transaction and shipment to a customer, both without further customer action being required. For example, upon approval by the veterinarian, an approval record of prescription product 402 is sent to the backend system without further input or interaction by the user.

As illustrated in FIG. 4C, order confirmation user interface 518 may display a confirmation to the user that the purchase of prescription product 402 is completed. Order confirmation user interface 518 may include confirmation prescription product 402, shipping address 412, billing address 414, quantity requested 413, and price 416 of prescription product 402.

In some embodiments, an ordering authorized user (e.g., veterinarian) may be required to approve the order for the prescription product, such as when the order is placed by a customer that is not an ordering authorized user (e.g., veterinarian) of the backend system. Referring to FIG. 4D-4E, there are shown exemplary approval user interfaces 541-542 for approving an order by an ordering authorized user. In some embodiments, a push notification is transmitted to a mobile device of the ordering authorized user. In FIG. 4D, the approval user interface 541 includes a prescription information object 550 that includes ordering information for prescription product 402 submitted by a customer using order confirmation user interface 518 of FIG. 4C. The approval user interface 541 also includes a prescription approval object 552 that includes an approval affordance 540. By default, the approval affordance 540 is not selected (e.g., unchecked). In response to an indication of approval by, e.g., selecting the approval affordance 540, approval affordance 540 may change to indicate that the prescription has been approved. For example, in FIG. 4E, approval affordance 540 has a check box 541a and approval text 541b which may indicate approval of the order by the veterinarian. The prescription approval information may then be submitted to the backend system for further processing with the order.

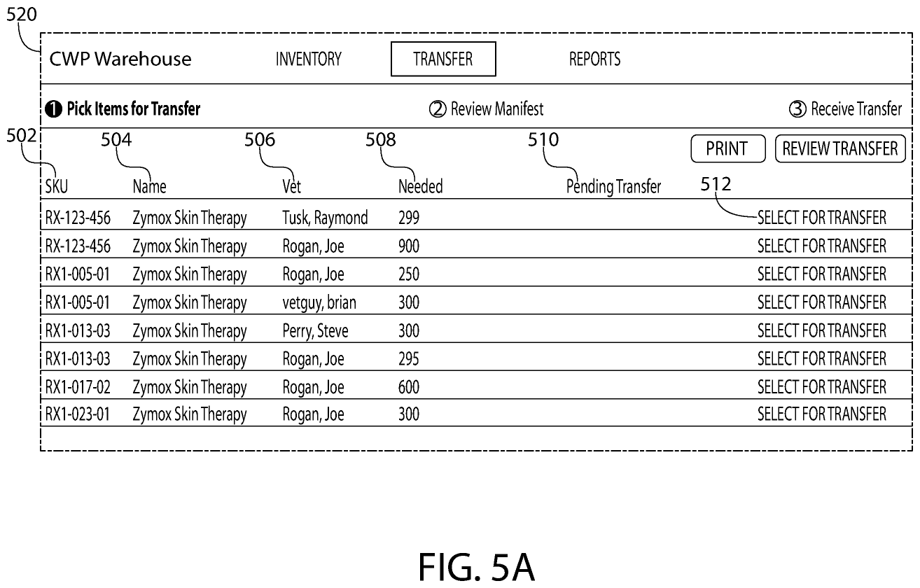

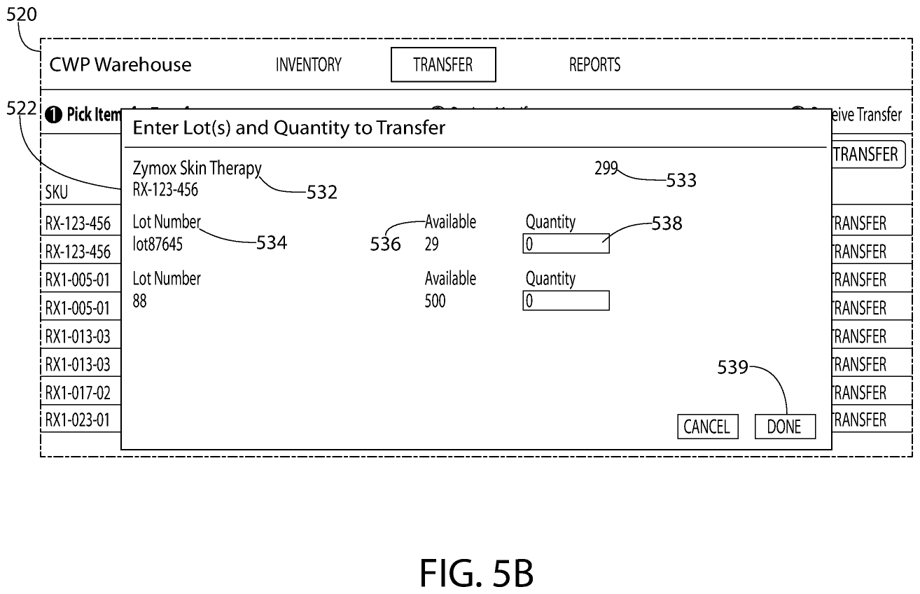

In an embodiment, only after the user has completed purchase of prescription product 402 and the purchase of prescription product 402 is approved, would order information associated with the purchase be sent to a central pharmacy OMS 106 and/or wholesale pharmacy 110 associated with a wholesale warehouse storing prescription product 402. The warehouse may then transfer a desired quantity of prescription product 402 to a retailer for shipment to the customer or veterinarian. The system may include product transfer user interface 520 for a warehouse administrator to manage product inventory transfer between the warehouse associated with the wholesale pharmacy server 110 and the warehouse associated with the retail pharmacy 108, illustrated in FIGS. 5A-5B. Product transfer user interface 520 may display information regarding the present inventory of various prescription products within the warehouse. Product user interface 520 may display stock keeping unit (SKU) 502, product name 504, veterinarian name/identification information 506 associated with the order, quantity needed 508, and status of transfer 510. In an embodiment, product transfer user interface 520 may display the total quantity of prescription product 402 ordered (as shown in FIG. 4C) and a veterinarian associated with prescription product 402. Product transfer user interface 520 may display the total quantity of a specific prescription product that is associated with a specific veterinarian. By way of an example, as illustrated in FIG. 5A, the total amount of “Zymox Skin Therapy” associated with veterinarian “Dr. Raymond Tusk” that is ordered and thus needed is “299,” and the total amount of “Zymox Skin Therapy” associated with veterinarian “Dr. Joe Rogan” that is ordered and thus needed is “900.” The quantity that is ordered and needed is the amount that is required to be sent from the warehouse to the retailer. To select an item for transfer to fulfill the required order, an administrator may select transfer request 512.

As illustrated in FIG. 5B, in response to an administrator selecting transfer request 512, product transfer user interface 520 may display transfer user interface object 522. Transfer user interface object 522 may be used to enter the quantity of prescription product 532 that is required to be transferred from the warehouse to the retailer. Transfer user interface object 522 may include quantity required 533 of prescription product 532, lot number 534, and quantity available 536 of prescription product 532. Quantity available 536 of prescription product 532 may indicate the quantity of prescription product 532 available corresponding to specific lot number in the warehouse. Each lot number 534 may include a specific quantity of prescription product 532. Transfer user interface object 522 may further include quantity input field 538. Quantity input field 538 may receive a numerical input from an administrator. The numerical input inputted into quantity input field 538 may indicate the amount of prescription product 532 required to be transferred from the warehouse to the retailer. The quantity inputted into quantity input field 538 may be determined based on quantity required 533 of prescription product 532. For example, as illustrated in FIG. 5B, in viewing transfer user interface object 522, an administrator is notified that 299 units of Zymox Skin Therapy need to be transferred from the warehouse to a retailer. The administrator is also notified that Lot Number lot87645 contains “29” units of Zymox Skin Therapy and Lot Number “88” contains “500” units of Zymox Skin Therapy. Therefore, the administrator may select “29” units from Lot Number lot87645 and “270” units from Lot Number “88” to obtain the required quantity of “299” units. Alternatively, the administrator may also select “20” units from Lot Number lot87645 and “279” units from Lot Number “88” to obtain the required quantity of “299” units. To complete the transfer of prescription product 532 from wholesale to retailer, the administrator may select completion button 539.

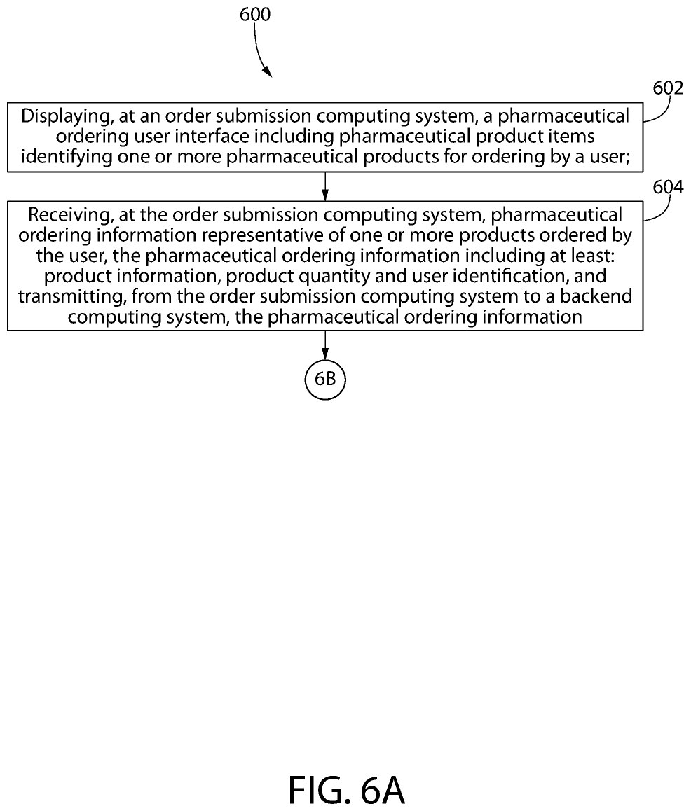

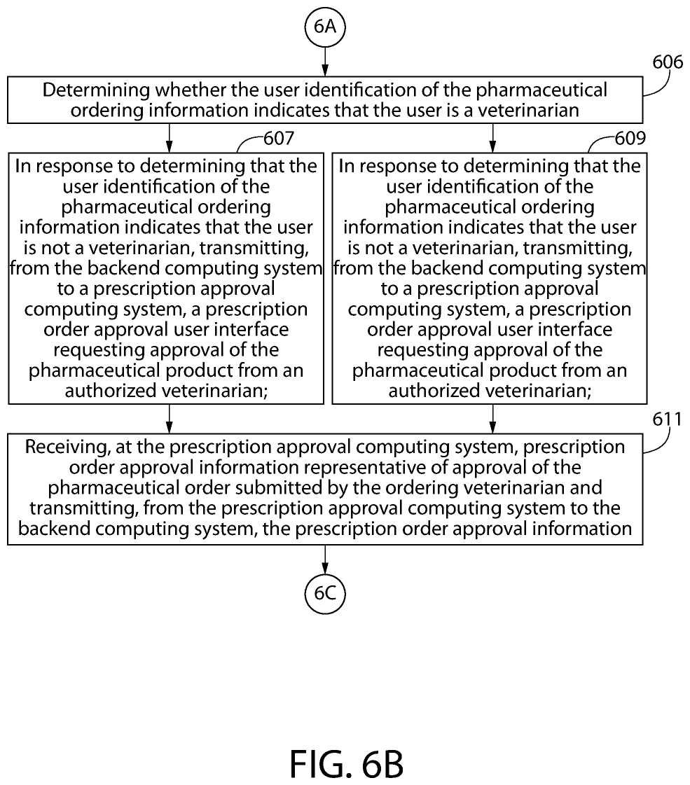

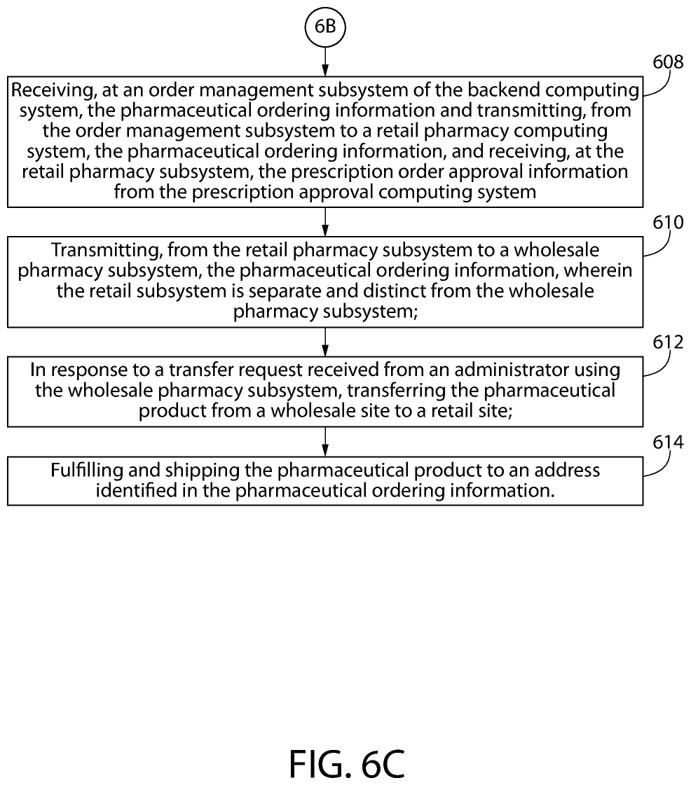

FIGS. 6A-6C illustrates a flow diagram of an exemplary method 600 of the present invention. Referring to FIG. 6A, in an embodiment, method 600 may comprise step 602 of displaying, at an order submission computing system (e.g., veterinarian server 102 of FIG. 1), a pharmaceutical ordering user interface including pharmaceutical product items identifying one or more pharmaceutical products for ordering by a user. For example, as shown in FIG. 4A, display product list user interface 514 may correspond to the pharmaceutical ordering user interface. In FIG. 4A, display product list user interface 514 is configured for displaying pharmaceutical products, such as pharmaceutical product 402, for ordering by a user.

In an embodiment, method 600 may comprise step 604 of receiving, at the order submission computing system, pharmaceutical ordering information representative of one or more products ordered by the user, the pharmaceutical ordering information including at least: product information, product quantity and user identification, and transmitting, from the order submission computing system to a backend computing system, the pharmaceutical ordering information. For example, as illustrated in FIG. 4C, pharmaceutical ordering information presented within order confirmation user interface 518 is initially received at the veterinarian server 102. The pharmaceutical ordering information may include the name of prescription product 402, quantity requested 413, price 416 of prescription product, and user identification such as shipping address 412 and billing address 414. In some embodiments, pharmaceutical ordering information may include pet information, prescription information, billing information and/or shipping information.

Referring to FIG. 6B, in an embodiment, method 600 may comprise step 606 of determining whether the user identification of the pharmaceutical ordering information indicates that the user is an ordering authorized user.

In an embodiment, step 607 includes, in response to determining that the user identification of the pharmaceutical ordering information indicates that the user is not an ordering authorized user, step 607 includes transmitting, from the backend computing system to a prescription approval computing system, a prescription order approval user interface requesting approval of the pharmaceutical product from an authorized veterinarian. FIG. 4D-4E show exemplary approval user interfaces 541-542 for approving an order by an ordering authorized user.

In an embodiment, step 609 includes, in response to determining that the user identification of the pharmaceutical ordering information indicates that the user is an ordering authorized user, step 609 includes refrain from transmitting, by the backend computing system to the prescription approval computing system, the prescription order approval user interface. For example, prior to completing the purchase of prescription product 402, if the user ordering the product is a non-veterinarian, a notification may be sent to a veterinarian associated with the user requesting approval of the veterinarian for purchasing of pharmaceutical product 402 by the user.

In an embodiment, step 611 includes receiving, at the prescription approval computing system (e.g., veterinarian device 101a), prescription order approval information representative of approval of the pharmaceutical order submitted by the ordering veterinarian and transmitting, from the prescription approval computing system to the backend computing system (e.g., central pharmacy OMS 106), the prescription order approval information.

Referring to FIG. 6C, in an embodiment, method 600 may comprise step 608 of receiving, at an order management subsystem of the backend computing system (e.g., central pharmacy OMS 106), the pharmaceutical ordering information and transmitting, from the order management subsystem to a retail pharmacy computing system (e.g., retail pharmacy server 108), the pharmaceutical ordering information, and receiving, at the retail pharmacy subsystem, the prescription order approval information from the prescription approval computing system.

In an embodiment, method 600 may comprise step 610 of transmitting, from the retail pharmacy subsystem (e.g., retail pharmacy server 108) to a wholesale pharmacy subsystem (e.g., wholesale pharmacy 110), the pharmaceutical ordering information, wherein the retail pharmacy subsystem is separate and distinct from the wholesale pharmacy subsystem.

In an embodiment, method 600 may comprise step 612 of, in response to a transfer request received from an administrator using the wholesale pharmacy subsystem, transferring the pharmaceutical product from a wholesale site to a retail site. For example, as shown in FIGS. 5A-5B, transfer user interface 520 may include user interface object 522 for transferring pharmaceutical product 532 from a wholesale site to a retail site based on an administrator selecting transfer request 512.

In an embodiment, method 600 may comprise step 614 of fulfilling and shipping the pharmaceutical product to an address identified in the pharmaceutical ordering information. For example, based on the information shown in FIG. 4C, pharmaceutical product 402 may be shipped to shipping address 412.

In an embodiment, the pharmaceutical ordering information including the product information and veterinarian identification information is received at a wholesale pharmacy subsystem. The product information may include product name information and product quantity information. For example, as illustrated in FIG. 5A, transfer user interface 520 may display prescription product name 504 and veterinarian identification information such as veterinarian name 506 based on the pharmaceutical ordering information. In some embodiments, a wholesale database is created, at the wholesale pharmacy subsystem, the wholesale database including a wholesale pharmaceutical order record including: the veterinarian identification information, the product quantity and the product name information.

In an embodiment, a wholesale database with a respective product wholesale inventory record for one or more pharmaceutical products is populated. For each pharmaceutical product, the respective product wholesale inventory record including the product name and a product wholesale inventory count. For example, as illustrated in FIG. 5B, transfer user interface object 522 may include prescription/pharmaceutical product 532 and quantity available 536 for each lot number 534 based on the product wholesale inventory record. In some embodiments, a retail database with a respective product retail inventory record for one or more pharmaceutical products is populated. For each pharmaceutical product, the respective product retail inventory record including the product name and a product retail inventory count. In an embodiment, after transferring an amount of the pharmaceutical product from a wholesale site to a retail site in response to a transfer request from an administrator using the wholesale pharmacy subsystem, the respective product wholesale inventory record is updated by decrementing the product wholesale inventory count by the amount of the transferred pharmaceutical product, and the respective product retail inventory record is updated by incrementing the product retail inventory count by the amount of the transferred pharmaceutical product.

In an embodiment, determining whether the user identification of the pharmaceutical ordering information indicates that the user is an ordering authorized user includes comparing the user identification in the pharmaceutical ordering information to one or more veterinarian records in a veterinarian database. In response to detecting a match of the user identification to one of the veterinarian records, it is determined if the user identification of the pharmaceutical ordering information indicates that the user is an ordering authorized user. In response to failing to detect a match of the user identification to one of the veterinarian records, it is determined if the user identification of the pharmaceutical ordering information indicates that the user is not a veterinarian.

In an embodiment, before transferring the pharmaceutical product from a wholesale site to a retail site in response to a transfer request from an administrator using the wholesale pharmacy subsystem, a warehouse inventory transfer user interface is displayed, including one or more pharmaceutical product order items, each warehouse pharmaceutical product order item includes a pharmaceutical product name, a veterinarian identification indicating the veterinarian associated with a respective pharmaceutical product order, a pharmaceutical quantity, and a transfer approval affordance that, when selected by the administrator, generates the transfer request from the administrator. For example, as illustrated in FIG. 5A, transfer user interface 520 may display the product name 504, veterinarian identification information 506, quantity needed 508, status of transfer 510, and transfer request 512. When transfer request 512 is selected by an administrator, transfer user interface object 522 is displayed to generate a transfer request.

In an embodiment, the prescription order approval information comprises authorization information unique to the authorizing veterinarian or the ordering veterinarian. In another embodiment, the prescription order approval information consists essentially of authorization information unique to the authorizing veterinarian or the ordering veterinarian.

In an embodiment, the pharmaceutical ordering information includes auto-shipment information. The auto shipment information may include a number of future auto shipments and at least one of a frequency of auto shipments and date information. In an embodiment, an order is created associated with a single auto shipment event at a predetermined time relative to a schedule associated with the auto shipment event, and creating a next order is delayed, where the next order is associated with a future single auto shipment event until a predetermined time relative to a schedule that the future auto shipment is due to be made. For example, as illustrated in FIG. 4B, selection confirmation user interface 516 may include repeat delivery option 408. Repeat delivery option 408 may allow a user to select the frequency at which prescription product 402 will be automatically delivered. For example, a user may select every two weeks as the selection for repeat delivery option 408, therefore prescription product would be automatically delivered to the user every two weeks. For orders requiring auto-shipment, a start date is generated and associated with the order when the order is created to track when shipments should occur. In some embodiments, the start date is generated and stored with other product order information when the order is created and not when an order is approved by an authorized user, as described herein.

In an embodiment, an electronic consignment record indicative of a wholesale quantity of the one or more pharmaceutical products and a retail quantity of the one or more pharmaceutical products is created. The retail quantity may reflect a quantity associated with an order.

It will be appreciated by those skilled in the art that changes could be made to the exemplary embodiments shown and described above without departing from the broad inventive concepts thereof. It is understood, therefore, that this invention is not limited to the exemplary embodiments shown and described, but it is intended to cover modifications within the spirit and scope of the present invention as defined by the claims. For example, specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”.

It is to be understood that at least some of the figures and descriptions of the invention have been simplified to focus on elements that are relevant for a clear understanding of the invention, while eliminating, for purposes of clarity, other elements that those of ordinary skill in the art will appreciate may also comprise a portion of the invention. However, because such elements are well known in the art, and because they do not necessarily facilitate a better understanding of the invention, a description of such elements is not provided herein.

Further, to the extent that the methods of the present invention do not rely on the particular order of steps set forth herein, the particular order of the steps should not be construed as limitation on the claims. Any claims directed to the methods of the present invention should not be limited to the performance of their steps in the order written, and one skilled in the art can readily appreciate that the steps may be varied and still remain within the spirit and scope of the present invention.

1. A computer-implemented method, the method comprising the steps of:
populating a database, at a backend computing system, first product data identifying one or more products for display on a veterinarian customer-facing user interface, the first product data including, for each product identified in the first product data:
product identifier,
product name,
product wholesale price;
transmitting, from the backend computing system, the first product data to a veterinarian computing system;
for each respective product identified in the first product data:
creating, at the veterinarian computing system, a respective product record at a veterinarian database at the veterinarian computing system, each respective product record including:
the product identifier,
the product name,
a product retail price indicative of a price of the respective product on the veterinarian customer-facing user interface, and
a product offering flag indicative of whether the veterinarian intends to offer the respective product on the veterinarian customer-facing user interface;
displaying, at a veterinarian administrative user interface of the veterinarian computing system, for each respective product record, a respective administrative product display item, the respective administrative product display item including:
the product name,
a product price field populated with the product retail price of the respective product record, and
a product offering field associated with the product offering flag and indicative of whether the veterinarian intends to offer the product on the veterinarian customer-facing user interface;
receiving, at the veterinarian administrative user interface, a user input at the product offering field of the respective administrative product display item;
determining whether the user input at the product offering field for the respective administrative product display item indicates user intent to offer the respective product on the veterinarian customer-facing user interface;
and
in response to determining the user intends to forego offering the product on the veterinarian customer-facing user interface, updating the product offering flag in the respective product record to indicate that the veterinarian intends to forego offering the product on the veterinarian customer-facing user interface.

2. The method of claim 1, further comprising:
receiving a request for displaying the veterinarian customer-facing user interface;
for each respective product record:
determining whether the respective product record includes the product offering field indicating user intent to offer the product,
in response to a determination that the respective product record includes the product offering field indicating user intent to offer the product, updating a veterinarian customer-facing user interface file representative of the veterinarian customer-facing user interface to include a customer product display item including:
the product name, and
a product price field populated with the product retail price of the respective product record; and
in response to a determination that the respective product record does not include the product offering field indicating user intent to offer the product, forego updating a veterinarian customer-facing user interface file representative of the veterinarian customer-facing user interface to include the customer product display item.

3. The method of claim 1, further comprising:
receiving, at the backend computing system, second product data identifying one or more products for display on the veterinarian customer-facing user interface;
transmitting, at the backend computing system, the second product data to the veterinarian computing system;
for each respective product identified in the second product data:
creating, at a veterinarian computing system, a respective product record at the veterinarian database at the veterinarian computing system.

4. The method of claim 1, further comprising:
receiving, at the veterinarian administrative user interface, a user input at the product price field of the respective administrative product display item, the user input including a price indicative of the price the veterinarian intends to sell the product on the veterinarian customer-facing user interface; and
updating the product price in the respective product record to the price indicated in the product price field.

5. The method of claim 1, wherein the product retail price is initially set to the product wholesale price of the respective product.

6. A system comprising:
one or more memory units each operable to store at least one program; and
at least one processor communicatively coupled to the one or more memory units, in which the at least one program, when executed by the at least one processor, causes the at least one processor to perform the steps of:
populating a database, at a backend computing system, first product data identifying one or more products for display on a veterinarian customer-facing user interface, the first product data including, for each product identified in the first product data:
product identifier,
product name,
product wholesale price;
transmitting, from the backend computing system, the first product data to a veterinarian computing system;
for each respective product identified in the first product data:
creating, at the veterinarian computing system, a respective product record at a veterinarian database at the veterinarian computing system, each respective product record including:
the product identifier,
the product name,
a product retail price indicative of a price of the respective product on the veterinarian customer-facing user interface, and
a product offering flag indicative of whether the veterinarian intends to offer the respective product on the veterinarian customer-facing user interface;
displaying, at a veterinarian administrative user interface of the veterinarian computing system, for each respective product record, a respective administrative product display item, the respective administrative product display item including:
the product name,
a product price field populated with the product retail price of the respective product record, and
a product offering field associated with the product offering flag and indicative of whether the veterinarian intends to offer the product on the veterinarian customer-facing user interface;
receiving, at the veterinarian administrative user interface, a user input at the product offering field of the respective administrative product display item;
determining whether the user input at the product offering field for the respective administrative product display item indicates user intent to offer the respective product on the veterinarian customer-facing user interface;
and
in response to determining the user intends to forego offering the product on the veterinarian customer-facing user interface, updating the product offering flag in the respective product record to indicate that the veterinarian intends to forego offering the product on the veterinarian customer-facing user interface.

7. A computer-implemented method, comprising:
displaying, at an order submission computing system, a pharmaceutical ordering user interface including pharmaceutical product items identifying one or more pharmaceutical products for ordering by a user;
receiving, at the order submission computing system, pharmaceutical ordering information representative of one or more products ordered by the user, the pharmaceutical ordering information including at least: product information, product quantity and user identification;
transmitting, from the order submission computing system to a backend computing system, the pharmaceutical ordering information;
determining whether the user identification of the pharmaceutical ordering information indicates that the user is authorized to approve a prescription;
in response to determining that the user identification of the pharmaceutical ordering information indicates that the user is not authorized to approve a prescription, transmitting, from the backend computing system to a prescription approval computing system, a prescription order approval user interface requesting approval of the pharmaceutical product from an authorized veterinarian;
in response to determining that the user identification of the pharmaceutical ordering information indicates that the user is authorized to approve a prescription, refrain from transmitting, by the backend computing system to the prescription approval computing system, the prescription order approval user interface;
receiving, at the prescription approval computing system, prescription order approval information indicative of approval of the pharmaceutical order submitted by the authorized user;
transmitting, from the prescription approval computing system to the backend computing system, the prescription order approval information;
receiving, at an order management subsystem of the backend computing system, the pharmaceutical ordering information and transmitting, from the order management subsystem to a retail pharmacy computing system, the pharmaceutical ordering information;
receiving, at the retail pharmacy subsystem, the prescription order approval information from the prescription approval computing system;
in response to a transfer request received from an administrator using a wholesale pharmacy subsystem, transferring the pharmaceutical product from a wholesale site to a retail site; and
fulfilling and shipping the pharmaceutical product to an address identified in the pharmaceutical ordering information.

8. The method of claim 7, further comprising:
receiving, at the wholesale pharmacy subsystem, the pharmaceutical ordering information including the product information and veterinarian identification information, wherein the product information includes:
product name information and product quantity information; and
creating a wholesale database, at the wholesale pharmacy subsystem, a wholesale pharmaceutical order record including: the veterinarian identification information, the product quantity and the product name information.

9. The method of claim 7, further comprising:
populating a wholesale database with a respective product wholesale inventory record for one or more pharmaceutical products, wherein for each pharmaceutical product, the respective product wholesale inventory record including a product name and a product wholesale inventory count;
populating a retail database with a respective product retail inventory record for one or more pharmaceutical products, wherein for each pharmaceutical product, the respective product retail inventory record including the product name and a product retail inventory count;
after transferring an amount of the pharmaceutical product from a wholesale site to a retail site in response to a transfer request using the wholesale pharmacy subsystem:
updating the respective product wholesale inventory record by decrementing the product wholesale inventory count by the amount of the transferred pharmaceutical product; and
updating the respective product retail inventory record by incrementing the product retail inventory count by the amount of the transferred pharmaceutical product.

10. The method of claim 7, further comprising:
determining whether the user identification of the pharmaceutical ordering information indicates that the user is a ordering authorized user includes:
comparing the user identification in the pharmaceutical ordering information to one or more veterinarian records in a veterinarian database;
in response to detecting a match of the user identification to one of the veterinarian records, determining that the user identification of the pharmaceutical ordering information indicates that the user is authorized to approve a prescription; and
in response to failing to detecting a match of the user identification to one of the veterinarian records, determining that the user identification of the pharmaceutical ordering information indicates that the user is not authorized to approve a prescription.

11. The method of claim 7, before transferring the pharmaceutical product from a wholesale site to a retail site in response to a transfer request from an administrator using the wholesale pharmacy subsystem:
displaying a warehouse inventory transfer user interface including one or more pharmaceutical product order items, each warehouse pharmaceutical product order item includes:
a pharmaceutical product name,
a veterinarian identification indicating the veterinarian associated with a respective pharmaceutical product order,
a pharmaceutical quantity, and
a transfer approval affordance that, when selected by the administrator, generates the transfer request from the administrator.

12. The method of claim 7, wherein the prescription order approval information comprises authorization information unique to the authorizing veterinarian.

13. The method of claim 7, wherein pharmaceutical ordering information includes auto-shipment information.

14. The method of claim 13, wherein the auto shipment information comprises a number of future auto shipments and at least one of a frequency of auto shipments and date information.

15. The method of claim 13, further comprising:
creating an order associated with a single auto shipment event at a predetermined time relative to a schedule associated with the auto shipment event;
delaying creating a next order associated with a future single auto shipment event until a predetermined time relative to a schedule that the future auto shipment is due to be made.

16. The method of claim 7 further comprising:
creating an electronic consignment record indicative of a wholesale quantity of the one or more pharmaceutical products and a retail quantity of the one or more pharmaceutical products, the retail quantity reflecting a quantity associated with an order.

17. The method of claim 7 further comprising:
transmitting, from the retail pharmacy subsystem to a wholesale pharmacy subsystem, the pharmaceutical ordering information, wherein the retail subsystem is separate and distinct from the wholesale pharmacy subsystem;
in response to a transfer request received at the wholesale pharmacy subsystem, transferring the pharmaceutical product record from a wholesale site to a retail site.

18. A system comprising:
one or more memory units each operable to store at least one program; and
at least one processor communicatively coupled to the one or more memory units, in which the at least one program, when executed by the at least one processor, causes the at least one processor to perform the steps of:
displaying, at an order submission computing system, a pharmaceutical ordering user interface including pharmaceutical product items identifying one or more pharmaceutical products for ordering by a user;
receiving, at the order submission computing system, pharmaceutical ordering information representative of one or more products ordered by the user, the pharmaceutical ordering information including at least: product information, product quantity and user identification;
transmitting, from the order submission computing system to a backend computing system, the pharmaceutical ordering information;
determining whether the user identification of the pharmaceutical ordering information indicates that the user is authorized to approve a prescription;
in response to determining that the user identification of the pharmaceutical ordering information indicates that the user is not authorized to approve a prescription, transmitting, from the backend computing system to a prescription approval computing system, a prescription order approval user interface requesting approval of the pharmaceutical product from an authorized veterinarian;
in response to determining that the user identification of the pharmaceutical ordering information indicates that the user is authorized to approve a prescription, refrain from transmitting, by the backend computing system to the prescription approval computing system, the prescription order approval user interface;
receiving, at the prescription approval computing system, prescription order approval information indicative of approval of the pharmaceutical order submitted by the authorized user;
transmitting, from the prescription approval computing system to the backend computing system, the prescription order approval information;
receiving, at an order management subsystem of the backend computing system, the pharmaceutical ordering information and transmitting, from the order management subsystem to a retail pharmacy computing system, the pharmaceutical ordering information;
receiving, at the retail pharmacy subsystem, the prescription order approval information from the prescription approval computing system;
in response to a transfer request received from an administrator using a wholesale pharmacy subsystem, transferring the pharmaceutical product from a wholesale site to a retail site; and
fulfilling and shipping the pharmaceutical product to an address identified in the pharmaceutical ordering information.
